(19) United States
(12) Patent Application Publication
(10) Pub. No.: US 2008/0039697 A1
(43) Pub. Date: Feb. 14, 2008

(54) Endoscope system

(75) Inventor: Koki Morishita, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS CORPORATION, Tokyo (JP)

(21) Appl. No.: 11/890,427

(22) Filed: Aug. 6, 2007

(30) Foreign Application Priority Data
Aug. 11, 2006 (JP) 2006-219420

(51) Int. Cl. A61B 1/06

(52) U.S. Cl. 600/181

(57) ABSTRACT

The invention provides an endoscope system, a portion of which is adapted to be inserted inside a body cavity of a living organism for acquiring images of an acquisition target inside the body cavity, including a light source unit for selectively emitting excitation light with different spectral characteristics; an image acquisition unit, having a filter which blocks each excitation light, and having sensitivity in a wavelength band of fluorescence; a storage unit for storing information regarding a relation between intensity of fluorescence generated and concentration of each fluorescent agent; and a concentration-information calculating unit for calculating and outputting concentration information of each fluorescent agent on the basis of the fluorescence intensity of images acquired by the image acquisition unit and the information regarding the relation stored in the storage unit.

### BACKGROUND OF THE INVENTION

1. Field of the Invention

The present invention relates to an endoscope system.

This application is based on Japanese Patent Application No. 2006-219420, the content of which is incorporated herein by reference.

2. Description of Related Art

In cancer cells, it is known that specific biological molecules or the like are overexpressed compared to normal cells. One proposed approach to diagnose cancer cells is to make expressed protein molecules or the like fluorescent by using a fluorescent probe, and to identify cancer cells by observing this fluorescence endoscopically (for example, see Japanese Unexamined Patent Application, Publication No. H10-201707).

In Japanese Unexamined Patent Application, Publication No. H10-201707, an endoscope apparatus for diagnosing cancer cells using one type of fluorescent probe is disclosed.

However, the molecule overexpressed in cancer cells sometimes is overexpressed also in inflamed sections, benign tumors, and so forth. Therefore, using one type of fluorescent probe has the drawback that the diagnostic ability for identifying cancer cells is low.

On the other hand, it is known that there are numerous biological molecules overexpressed in cancer cells. Thus, the diagnostic ability can be improved by making plural types of molecules related to these cancer cells fluorescent using fluorescent dyes each with different optical characteristics to observe those molecules.

However, when plural types of fluorescent agents are observed, there is a problem of mixing of the fluorescence. Since fluorescence generated by exciting fluorescent agents is very week, it is desirable to acquire fluorescence in a wide wavelength band. However, using two or more types of fluorescent agents makes the wavelength bands of the fluorescence overlap, and therefore has the drawback that no distribution images of each fluorescent agent can be acquired, but only images exhibiting mixed fluorescence. The influence of this fluorescence mixing can be reduced by observing the fluorescence of each agent with a filter optimized for the fluorescent characteristics of each fluorescent agent. However, it is difficult to install a system for replacing filters in a videoscope having a CCD (Charge Coupled Device) at the tip of the endoscope.

### BRIEF SUMMARY OF THE INVENTION

The present invention provides the following solutions.

According to an aspect of the present invention, an endoscope system, at least a portion of which is adapted to be inserted inside a body cavity of a living organism for acquiring images of an acquisition target inside the body cavity, includes a light source unit for selectively emitting two or more types of excitation light with different spectral characteristics in order to excite two or more types of fluorescent agents with different optical characteristics; an image acquisition unit disposed in the portion that is adapted to be inserted inside the body cavity, having a filter which blocks each excitation light, and having sensitivity in a wavelength band of two or more types of fluorescence emitted from the acquisition target by each type of excitation light; a storage unit for storing information regarding a relation between intensity of fluorescence generated when the fluorescent agent is excited by each type of excitation light and concentration of each fluorescent agent; and a concentration-information calculating unit for calculating and outputting concentration information of each fluorescent agent on the basis of the intensity of fluorescence of two or more images acquired by the image acquisition unit and the information regarding the relation stored in the storage unit. Additionally, the information regarding the relation may be about a proportion of the intensity of fluorescence generated when the fluorescent agent is excited by each type of excitation light and the concentration of each fluorescent agent.

In the aspect described above, the endoscope system may include a display unit for displaying the concentration information which is calculated and output by the concentration-information calculating unit.

In the aspect described above, the display unit may include a plurality of channels corresponding to display colors. The concentration information corresponding to each fluorescent agent may be assigned to each channel and output.

Furthermore, in the aspect described above, a wavelength of each excitation light is preferably in the near infrared region or in a longer wavelength region than the near infrared region.

### BRIEF DESCRIPTION OF THE SEVERAL VIEWS OF THE DRAWINGS

FIG. 1 is a block diagram showing the overall structure of an endoscope system according to a first embodiment of the present invention.

FIG. 2 is a diagram showing the wavelength characteristics of an excitation-light cutting filter, excitation light, illumination light, and fluorescence generated by the excitation light in the endoscope system in FIG. 1.

FIG. 3 is a timing chart for explaining the operation of the endoscope system in FIG. 1.

FIG. 4 is a timing chart for explaining the operational states of a valve control circuit in the endoscope system in FIG. 1.

FIG. 5 is a diagram showing the wavelength characteristics of an excitation-light cutting filter, excitation light, illumination light, and fluorescence generated by the excitation light in the endoscope system in FIG. 1, using different fluorescent probes from the probes used in FIG. 2.

### DETAILED DESCRIPTION OF THE INVENTION

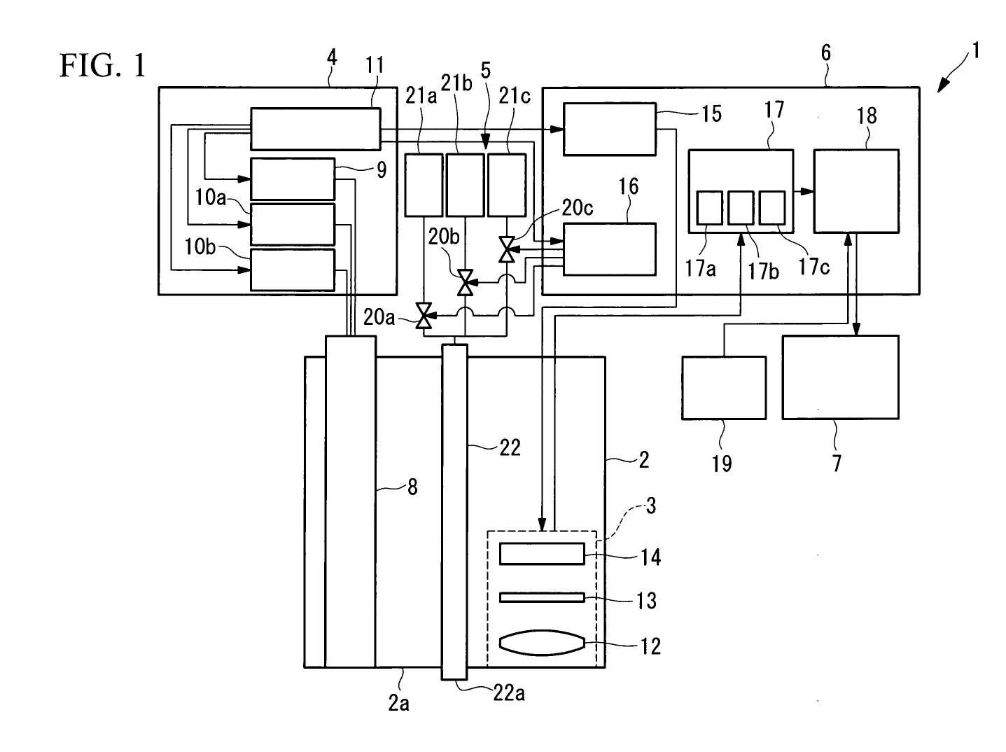

An endoscope system according to a first embodiment of the present invention will now be described with reference to FIGS. 1 to 5.

As shown in FIG. 1, the endoscope system 1 according to this embodiment includes an insertion portion 2 for inserting into a body cavity of a living organism; an image acquisition unit (image acquisition portion) 3 disposed inside the insertion portion 2; a light source unit (light source portion) 4 for emitting plural types of light; a liquid delivery unit (discharging portion) 5 for supplying liquid to be discharged from a tip 2a of the insertion portion 2; a control unit 6 for controlling the image acquisition unit 3, the light source unit 4, and the liquid delivery unit 5; and a display unit (display portion) 7 for displaying images acquired by the image acquisition unit 3.

The insertion portion 2 has extremely narrow outer dimensions, so that it can be inserted inside the body cavity of the living organism. Additionally, the insertion portion 2 includes the image acquisition unit 3 and a light guide (optical system) 8 for transmitting light from the light source unit 4 to the tip 2a.

The light source unit 4 includes an illumination light source (light source portion) 9 which emits illumination light (irradiation light) for illuminating an observation target inside the body cavity to obtain reflected light returning after reflection at the observation target, two excitation light sources (light source portions) 10a and 10b which emits two types of excitation light for irradiating the observation target inside the body cavity to generate fluorescence upon exciting a fluorescent material present inside the observation target, and a light source control circuit (control portion) 11 for controlling these light sources 9, 10a, and 10b.

The illumination light source 9 is, for example, a combination of a xenon lamp (not shown) and color filters which are switched sequentially. The color filters sequentially generate red (R), green (G), and blue (B) illumination light.

The excitation light source 10a is, for example, a semiconductor laser emitting first excitation light with a peak wavelength of 680±5 nm. The first excitation light can excite a fluorescent probe based on Alexa Fluor (registered trademark of Invitrogen) 680. The excitation light source 10b is, for example, a semiconductor laser emitting second excitation light with a peak wavelength of 700±5 nm. The second excitation light can excite a fluorescent probe based on Alexa Fluor® 700.

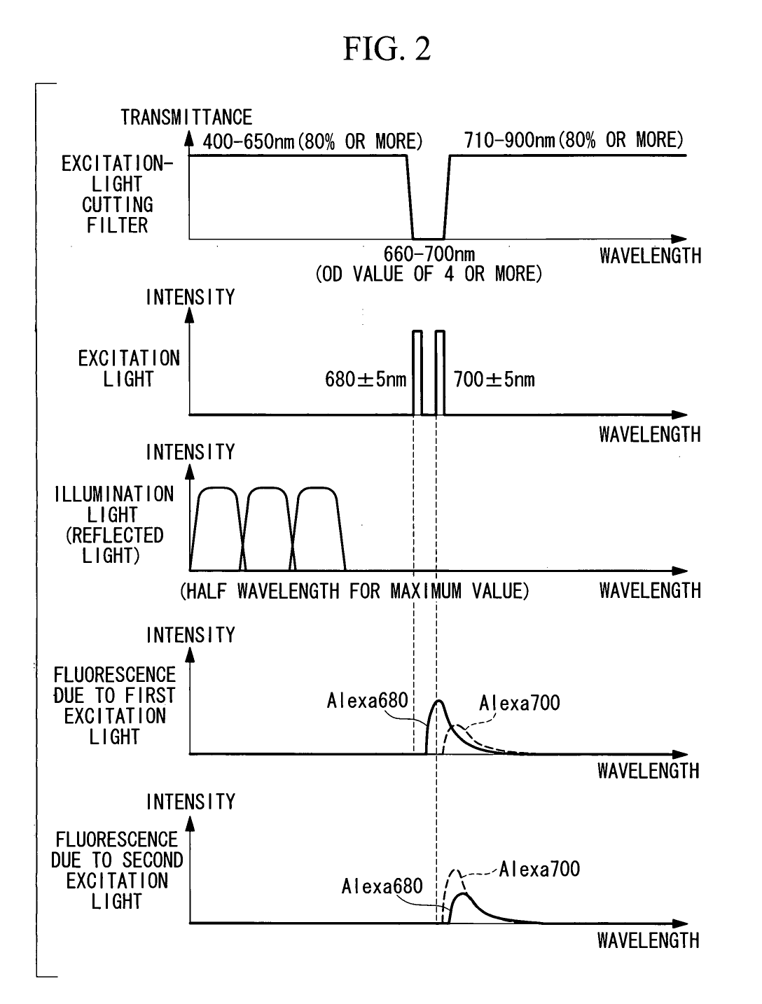

As shown in FIG. 2, wavelength bands of fluorescence generated by exciting Alexa Fluor® 680 and Alexa Fluor® 700 are overlapped. Thus, when the observation target is irradiated by one of the first excitation light and the second excitation light while these two fluorescent probes are applied to the observation target, the two fluorescent probes are simultaneously excited. As a result, fluorescence is emitted by the two different types of fluorescent probes at the same time.

The light source control circuit 11 is configured to alternately turn on and off the illumination light source 9 and the excitation light sources 10a and 10b at a predetermined timing according to a timing chart to be described later.

The image acquisition unit 3 includes an image acquisition optical system 12 for collecting light incident from the observation target A, an excitation-light cutting filter 13 for blocking the excitation light incident from the observation target A, and an image acquisition device (image acquisition portion) 14 for acquiring the light collected by the image acquisition optical system 12 and converting the light to an electrical signal. The image acquisition device 14 is adapted to have sensitivity in a wide wavelength band of 400 nm to 900 nm.

The excitation-light cutting filter 13 has transmittance characteristics exhibiting a transmittance of 80% or more in the wavelength band of 400 to 650 nm, an OD value of 4 or more (=a transmittance of 1×10⁻⁴ or less) in the wavelength band of 660 to 700 nm, and a transmittance of 80% or more in the wavelength band of 710 to 900 nm.

As shown in FIG. 1, the control unit 6 includes an image-acquisition-device driving circuit 15 for driving and controlling the image acquisition device 14, a valve control circuit 16 which is described later, a frame memory 17 for storing image information acquired by the image acquisition device 14, and an image processing circuit (storage unit, concentration-information calculating unit) 18 for processing the image information stored in the frame memory 17 and outputting the image information to the display unit 7.

Additionally, an input unit 19 is connected to the image processing circuit 18.

The image-acquisition-device driving circuit 15 and the valve control circuit 16 are connected to the light source control circuit 11 and synchronize the driving control of the image acquisition device 14 and valves 20a, 20b, and 20c with the switching of the illumination light source 9 and the excitation light sources 10a and 10b by the light source control circuit 11.

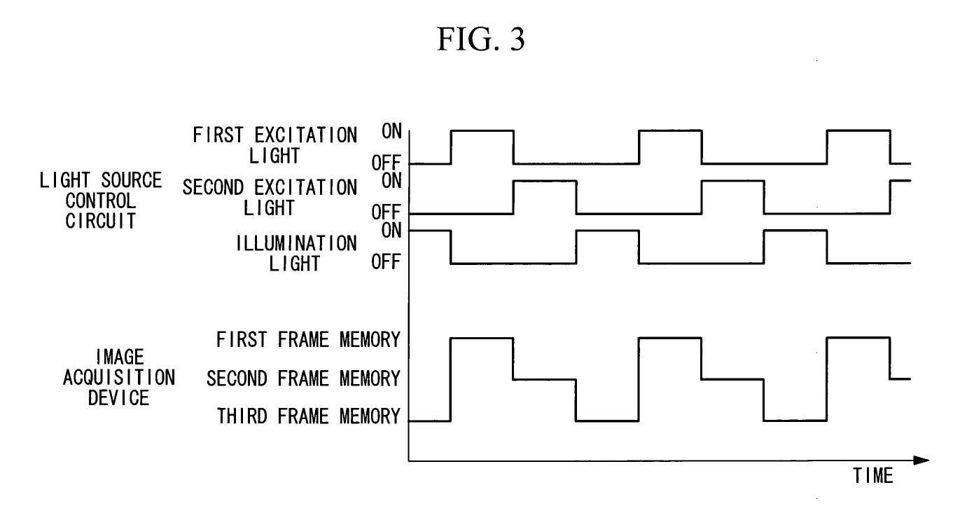

More concretely, as shown in the timing chart in FIG. 3, when the first excitation light is emitted by the excitation light source 10a according to the operation of the light source control circuit 11, the image-acquisition-device driving circuit 15 is configured to output the image information which is output from the image acquisition device 14 to a first frame memory 17a. In addition, when the second excitation light is emitted by the excitation light source 10b, the image-acquisition-device driving circuit 15 is configured to output the image information output from the image acquisition device 14 to a second frame memory 17b.

Furthermore, when illumination light is emitted by the illumination light source 9, the image-acquisition-device driving circuit 15 is configured to output the image information output from the image acquisition device 14 to a third frame memory 17c.

The image processing circuit 18 is configured to receive first fluorescence-image information acquired due to the emission of the first excitation light and second fluorescence-image information acquired due to the emission of the second excitation light from the first and the second frame memories 17a and 17b, respectively, and to perform the calculation process. The calculation process by the image processing circuit 18 is described below.

That is, fluorescence intensities per unit concentration acquired from the fluorescent probe based on Alexa Fluor® 680 and the fluorescent probe based on Alexa Fluor® 700 when the first excitation light is emitted are defined as a and b, respectively. The fluorescence intensities per unit concentration acquired from the fluorescent probe based on Alexa Fluor® 680 and the fluorescent probe based on Alexa Fluor® 700 when the second excitation light is emitted are defined as c and d, respectively.

The fluorescence intensity in a certain region due to the emission of the first excitation light is defined as P1. The fluorescence intensity in the same region due to the emission of the second excitation light is defined as P2. The concentrations of the fluorescent probes based on Alexa Fluor® 680 and Alexa Fluor® 700 are defined as N1 and N2, respectively. The relation is shown in the following formula (I).

$$( \begin{matrix}P 1 \\ P 2\end{matrix} ) = ( \begin{matrix}a & b \\ c & d\end{matrix} ) ( \begin{matrix}N 1 \\ N 2\end{matrix} ) \qquad ( 1 )$$

Since the fluorescence intensities P1 and P2 are the results of measurements, the concentrations N1 and N2 of each fluorescent probe can be calculated by assigning the fluorescence intensities P1 and P2 in the formula (I). Factors a, b, c, and d in the formula (I) can be obtained beforehand by measurements or the like and may be input to an calculation processing circuit by using the input unit 19.

As a result of the calculation, the concentrations N1 and N2 of each fluorescent probe are configured to be output to a first (for example, red) channel and a second (for example, green) channel of the display unit. Additionally, the image processing circuit 18 is configured to receive reflected-light image information acquired due to the emission of illumination light from the third frame memory 17c and to output it to a third (for example, blue) channel of the display unit 7.

The liquid delivery unit 5 includes a first tank 21a for storing cleaning water for cleaning the observation target; a second tank 21b for storing first fluorescent probe liquid; a third tank 21c for storing second fluorescent probe liquid; the valves 20a, 20b, and 20c for selectively supplying/stopping liquid from these tanks 21a, 21b, and 21c; a liquid delivery tube 22, connected to the valves 20a, 20b, and 20c, for supplying each of the liquids to the tip 2a along the insertion portion 2; and the valve control circuit 16, disposed inside the control unit 6, for controlling the valves 20a, 20b, and 20c. An end 22a of the liquid delivery tube 22 is disposed in the tip 2a of the insertion portion 2, allowing the delivered cleaning water or the fluorescent probe liquid to be applied to the observation target A. For the liquid delivery tube 22, a forceps channel provided in the insertion portion 2 may be used.

The valve control circuit 16 is connected to the light source control circuit 11. The light source control circuit 11 is configured to output switching commands for the valves 20a, 20b, and 20c to the valve control circuit 16 on the basis of the timing for switching the light sources.

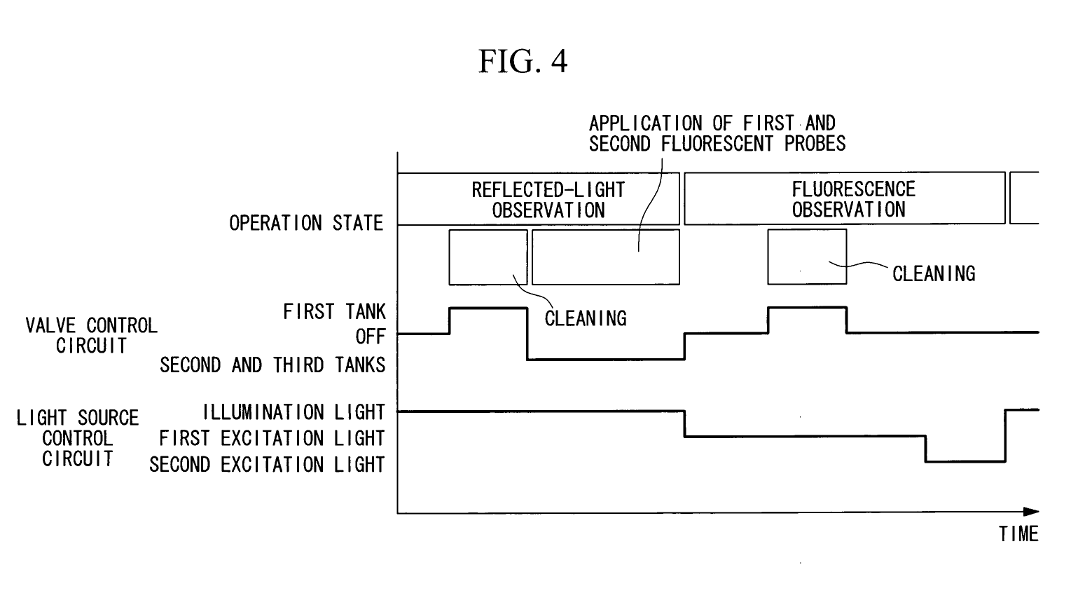

Accordingly, as shown in FIG. 4, while the observation target is observed with reflected light before a certain timing for switching to the first excitation light source 10a in response to the switching command from the light source control circuit 11, the valve control circuit 16 opens the valve 20a for a predetermined period of time and discharges the cleaning water stored in the first tank 21a. After discharging the cleaning water, the valve control circuit 16 closes the valve 20a and opens the valves 20b and 20c so that the fluorescent probe liquid stored in the second tank 21b and the third tank 21c may be applied.

In addition, the valve control circuit 16 switches the valves 20a, 20b, and 20c to the off state after applying the fluorescent probe liquid. Then, after a certain timing for switching to the first excitation light source 10a in response to the switching command from the light source control circuit 11, the valve control circuit 16 opens the valve 20a so that the cleaning water stored in the first tank 21a is discharged during a predetermined period of time. After discharging the cleaning water, the valve control circuit 16 is configured to close all the valves 20a, 20b and 20c.

The operation of the endoscope system 1 according to this embodiment, having such a configuration, will be described below.

To acquire an image of the acquisition target inside the body cavity of the living organism using the endoscope system 1 according to this embodiment, first, the insertion portion 2 is inserted into the body cavity so that the tip 2a thereof opposes the acquisition target in the body cavity. In this state, the light source unit 4 and the control unit 6 are operated, and by operating the light source control circuit 11, the illumination light source 9 and the excitation light sources 10a and 10b are sequentially operated to generate illumination light, the first excitation light, and the second excitation light, respectively.

In reflected light observation carried out with the emission of illumination light, the cleaning operation is carried out while checking the position to be cleaned using reflected light. After the cleaning operation, two types of fluorescent probe liquid are applied. After applying the two types of fluorescent probe liquid, the mode is switched from reflected light observation to fluorescence observation. The application of the fluorescent probes is checked using the fluorescence after applying the two types of fluorescent probes. After cleaning the applied area, fluorescence observation of the applied area is carried out.

The illumination light, the first excitation light, and the second excitation light generated in the light source unit 4 are transmitted to the tip 2a of the insertion portion 2 with the light guide 8 and emitted to the acquisition target from the tip 2a of the insertion portion 2.

In the case of the first excitation light irradiating the acquisition target, the two types of fluorescence probes permeate the acquisition target and are excited at the same time. As shown in the FIG. 2, two types of fluorescence are emitted from the acquisition target at the same time. The two types of fluorescence emitted from the acquisition target are collected by the image acquisition optical system 12 of the image acquisition unit 3, pass through the excitation light cutting filter 13, and are acquired by the image acquisition device 14.

Since the image acquisition device 14 is adapted to be sensitive to light over a wide wavelength band of 400 to 900 nm, overlapping fluorescence generated by the two types of fluorescent probes is acquired by the image acquisition device 14. As a result, fluorescence-image information with mixed fluorescence is acquired.

In this case, part of the first excitation light which irradiates the acquisition target is reflected at the acquisition target and is incident on the image acquisition unit 3 together with the fluorescence. However, because the excitation-light cutting filter 13 is provided in the image acquisition unit 3, the first excitation light is blocked and prevented from being incident on the image acquisition device 14.

The fluorescence-image information acquired by the image acquisition device 14 is stored in the first frame memory 17a.

Next, also in the case of the second excitation light which irradiates the acquisition target, the two types of fluorescent probes permeating the acquisition target are excited. As shown in the FIG. 2, fluorescence is emitted. The fluorescence emitted from the acquisition target is collected by the image acquisition optical system 12 of the image acquisition unit 3, passes through the excitation-light cutting filter 13, and is acquired by the image acquisition device 14.

Also in this case, although fluorescence-image information with mixed fluorescence, in which the two types of fluorescence emitted by the two types of fluorescent probes are overlapped, is acquired by the image acquisition device 14, the second excitation light returning after reflection at the acquisition target is blocked by the excitation-light cutting filter 13 and is prevented from being incident on the image acquisition device 14.

The fluorescence-image information acquired by the image acquisition device 14 is stored in the second frame memory 17b.

At this point, the image processing circuit 18 receives the fluorescence-image information due to the emission of the first and the second excitation light from the first and the second frame memories 17a and 17b. The image processing circuit 18 carries out the calculation based on formula (I) and calculates the concentrations N1 and N2 of the fluorescent probes based on Alexa Fluor® 680 and 700, respectively.

According to the endoscope system 1 of this embodiment, individual concentration information for each fluorescent probe can be calculated on the basis of the fluorescence-image information acquired in mixed fluorescence state. Accordingly, without using a special device like a variable-spectrum device, it is possible to easily observe the molecular distribution related to cancer cells using each fluorescent probe, on the basis of fluorescence in wavelength bands which overlap or which are too close to spectrally resolve even with fine control of a variable-spectrum device.

Information about the concentrations N1 and N2 calculated by the image processing circuit 18 is output to the first and the second channels of the display unit 7, respectively, and is displayed on the display unit 7.

Accordingly, an individual image which shows the molecular distribution related to cancer cells using each fluorescent probe is displayed on the display unit 7 in an overlapped form.

As a result, in the case of the fluorescence generated by the two fluorescent probes in the same region, it is easy to recognize that there is a high possibility of the existence of cancer cells in the region. In the region where fluorescence is generated by only one fluorescent probe, it is possible to judge that there is a low possibility of the existence of cancer cells. Thus, the present invention provides an advantage in that it is possible to improve the diagnostic ability by using two types of fluorescent probes at the same time.

In the case of illumination light irradiating the acquisition target, the illumination light is reflected at the surface of the acquisition target, is collected by the image acquisition optical system 12, and passes through the excitation-light cutting filter 13. Reflected light which passes through the excitation-light cutting filter 13 is incident on the image acquisition device 14. Then reflected-light image information is acquired. The acquired reflected-light image information is stored in the third frame memory 17c, output to the third channel of the display unit 7 by the image processing circuit 18, and displayed on the display unit 7.

As a result, an image representing the actual appearance of the observation target by the illumination light can be overlapped and displayed together with images which show the molecular distribution related to cancer cells by each fluorescent probe. Accordingly, it is possible to observe a region which has a high possibility of the existence of cancer cells based on the actual appearance image.

In the endoscope system 1 according to this embodiment, as described above, the reflected-light observation is carried out prior to the fluorescence observation by the operation of the light source control circuit 11 and the valve control circuit 16. In the reflected-light observation, the light source control circuit 11 operates the illumination light source 9 to emit illumination light towards the acquisition target.

When the reflected-light observation is switched to the fluorescence observation, prior to the emission of the excitation light, while the illumination light source 9 is emitting the illumination light, the valve control circuit 16 opens the valve 20a so that the cleaning water stored in the first tank 21a is discharged to the observation target from an end 22a of the liquid delivery tube 22 to clean the surface of the observation target A.

In the above case, according to this embodiment, since the observation target is cleaned while the illumination light source 9 is emitting the illumination light, it is possible to easily check an affected area and to clean the area on which the fluorescent probe liquid is to be applied while checking the area.

Since the application of the fluorescent probe liquid is carried out while the illumination light source 9 is emitting illumination light, the second and the third valves 20b and 20c are opened while checking the position of the cleaned observation target. Thus, a small amount of the fluorescent probe liquid can be applied precisely to the requisite area so that the position of the observation target is not missed. As a result, the expensive fluorescent probe is not wasted.

Then, the first excitation light source 10a is operated by the light source control circuit 11 so that the first excitation light irradiates the observation target. The valve control circuit 16 switches the valves 20a, 20b, and 20c to the off state, upon receiving a signal from the light source control circuit 11.

In the above case, according to this embodiment, after the application of the fluorescent probe liquid, the first excitation light source 10a emits the excitation light before the cleaning operation. Thus, the application condition can be checked using the fluorescence, even if the fluorescent probe is transparent.

Additionally, in the endoscope system 1 according to this embodiment, the wavelength band of the two types of excitation light is in the near infrared region or in a longer wavelength region than the near infrared region. Thus, an advantage is afforded in that it is possible to acquire a clearer image, while preventing autofluorescence generation, without exciting an autofluorescent substance which is naturally present in the observation target.

The endoscope system 1 according to this embodiment is described in the case of using fluorescent probes based on Alex Flour® 680 and 700. However, instead of these probes, Cy5.5 (manufactured by Amersham Biosciences Corp.) and Cy7 (manufactured by Amersham Biosciences Corp.) may be used.

In the above case, for the excitation light source 10a, a semiconductor laser which emits the first excitation light with a peak wavelength of 660±5 nm may be used to excite a fluorescent probe, for example, based on Cy5.5. For the excitation light source 10b, a semiconductor laser which emits the second excitation light with a peak wavelength of 740±5 nm may be used to excite a fluorescent probe, for example, based on Cy7.

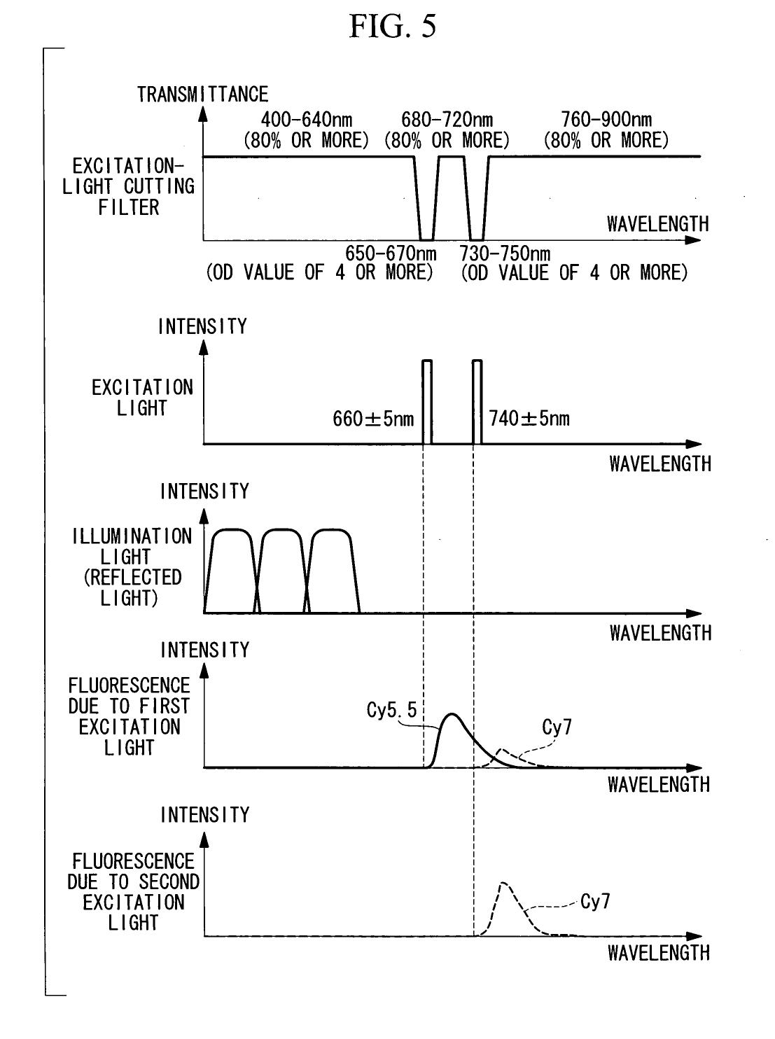

Additionally, for the excitation-light cutting filter, as shown in FIG. 5, it is possible to use a filter having transmittance characteristics exhibiting a transmittance of 80% or more in the wavelength band of 400 to 640 nm, an OD value of 4 or more (=a transmittance of 1×10⁻⁴ or less) in the wavelength band of 650 to 670 nm, a transmittance of 80% or more in the wavelength band of 680 to 720 nm, an OD value of 4 or more (=a transmittance of 1×10⁻⁴ or less) in the wavelength band of 730 to 750 nm, and a transmittance of 80% or more in the wavelength band of 760 to 900 nm.

By obtaining each value of the fluorescence intensity per unit concentration a, b, c, and d by measurement or the like in advance, it is possible to obtain the concentrations N1 and N2 of each fluorescent probe by using formula (1) even if these different types of fluorescent probes are used.

As shown in FIG. 5, the fluorescent probe Cy5.5 hardly generates any fluorescence in response to the second excitation light. Thus, the factor c can be set to 0.

Additionally, when defining the molecular distribution related to cancer cells using the fluorescent probe Cy7 as a reference, a specific molecular distribution from which the influence of the dynamics in the living organism due to the optical system and the fluorescent probe is excluded can be measured by calculating and displaying N1 and N2.

In this embodiment, by emitting the two types of light, that is, the excitation light and the illumination light, towards the observation target, the images which show the concentration distribution of fluorescent probes and the reflected-light image are overlapped and displayed. However, instead of the illumination light, third excitation light which excites autofluorescence in the observation target may be emitted.

Since autofluorescence has a wavelength band which is away from the near infrared region in which agent fluorescence is detected, autofluorescence can be detected without color mixing with agent fluorescence.

In the endoscope system 1 according to this embodiment, the diagnostic ability is improved by using the two types of fluorescent probes. Instead, three or more types of fluorescent probes may be used.

What is claimed is:

1. An endoscope system, at least a portion of which is adapted to be inserted inside a body cavity of a living organism for acquiring images of an acquisition target inside the body cavity, comprising:
a light source unit for selectively emitting two or more types of excitation light with different spectral characteristics in order to excite two or more types of fluorescent agents with different optical characteristics;
an image acquisition unit disposed in the portion that is adapted to be inserted inside the body cavity, having a filter which blocks each type of excitation light, and having sensitivity in a wavelength band of two or more types of fluorescence emitted from the acquisition target by each type of excitation light;
a storage unit for storing information regarding a relation between intensity of fluorescence generated when the fluorescent agent is excited by each type of excitation light and concentration of each fluorescent agent; and
a concentration-information calculating unit for calculating and outputting concentration information of each fluorescent agent on the basis of the fluorescence intensity of two or more images acquired by the image acquisition unit and the information regarding the relation stored in the storage unit.

2. An endoscope system according to claim 1, wherein the information regarding the relation is about a proportion of the intensity of fluorescence generated when the fluorescent agent is excited by each type of excitation light and the concentration of each fluorescent agent.

3. An endoscope system according to claim 1, further comprising a display unit for displaying the concentration information which is calculated and output by the concentration-information calculating unit.

4. An endoscope system according to claim 3, wherein the display unit comprises a plurality of channels corresponding to display colors, and the concentration information corresponding to each fluorescent agent is assigned to each channel and output.

5. An endoscope system according to claim 1, wherein a wavelength of each excitation light is in the near infrared region or in a longer wavelength region than the near infrared region.
